(19) United States
(12) Patent Application Publication
(10) Pub. No.: US 2017/0335066 A1
(43) Pub. Date: Nov. 23, 2017

(54) POLYMER COMPRISING ALKOXYSILANE GROUPS AND USE IN COSMETICS

(71) Applicant: L'OREAL, Paris (FR)

(72) Inventor: Pascal GIUSTINIANI, La Garenne Colombes (FR)

(21) Appl. No.: 15/522,819

(22) PCT Filed: Oct. 27, 2015
(86) PCT No.: PCT/EP2015/074807

§ 371 Date: Apr. 28, 2017

(30) Foreign Application Priority Data
Oct. 29, 2014 (FR) 1460393

(51) Int. Cl. C08G 77/62; A61Q 19/08; A61Q 3/02; A61K 8/58; A61K 8/898; A61K 8/87; C07F 7/18; A61Q 5/00

(52) U.S. Cl. CPC C08G 77/62; C07F 7/1836; A61Q 19/08; A61Q 5/00; A61K 8/585; A61K 8/898; A61K 8/87; A61Q 3/02

(57) ABSTRACT

The invention relates to a polymer comprising alkoxysilane groups, obtained by polycondensation of a diisocyanate, of a difunctional compound and of two different alkoxysilanes.

The invention also relates to a cosmetic composition comprising such a polymer and to a process for caring for or making up keratin materials by application of the composition to the keratin materials.

The present invention relates to a process for preparing a product containing polymeric compounds comprising an alkoxysilane group and to the use of such a product in a cosmetic composition for treating keratin materials, in particular the nails and the hair.

The use of sol/gel techniques for the purposes of preparing cosmetic compositions is known per se. Such compositions form films after application to keratin materials. After drying, a hybrid material is in fact formed by polycondensation and crosslinking.

For example, patent application WO 98/44906 describes a cosmetic or dermatological composition suitable for forming a coating on keratin materials via a reaction of sol/gel type obtained by mixing (A) at least one organometallic compound with (B) at least one functionalized organic polymer or at least one functionalized silicone polymer other than the first compound, and (C) an amount of water sufficient to hydrolyse the organometallic compound.

There is a need to provide compounds or compositions that have both stability properties before application thereof to keratin materials, and good reactivity.

There is also a need to have compounds which make it possible to obtain, after application thereof to keratin materials, a uniform and smooth deposit.

Furthermore, the layer of the composition deposited on the keratin materials is not tacky. It thus has a good appearance and is not degraded on contact with foreign bodies, for instance a glass, a cigarette, clothing or skin.

In addition, properties of persistence on washing with water and with detergents are sought.

It is sought to obtain a film which is resistant in particular to mechanical attacks such as rubbing, and is adherent.

Finally, sheen properties of the deposited film may be sought.

The inventors have found that such advantages may be obtained by using a polymeric product comprising particular alkoxysilane groups, as described hereinafter.

After application to keratin materials, the polymeric product, on contact with the moisture in the air, crosslinks to form a film. The film obtained is shiny and has good water resistance.

The polymeric product is thus suitable for use as a film-forming agent in a nail varnish composition or a hair composition, in particular for haircare or else for hair shaping (styling), or else in a composition for caring for or making up the skin, in particular for filling the surface irregularities of the skin, such as wrinkles or grooves.

More specifically, a subject of the present invention is a process for preparing a polymer comprising alkoxysilane groups (referred to as Pf) which can be obtained by polycondensation, comprising, in a first step, the reaction between:

(i) a diisocyanate of formula (I): OCN-Z-NCO (I)

in which Z denotes a divalent hydrocarbon-based radical containing from 4 to 20 carbon atoms; and

(ii) a difunctional compound of formula (II): H-T-A-T-H (II)

in which:

T denotes a heteroatom chosen from 0 and S or an —N(R)— radical, R being H or a C₁-C₄ alkyl radical, in particular methyl,

A denotes a linear or branched, divalent hydrocarbon-based C₂-C₁₀₀ radical, optionally interrupted with one or more non-adjacent heteroatoms chosen from 0 and S, or an —N(R′)— group in which R′ denotes a hydrogen atom or a C₁-C₄ alkyl radical, in particular methyl;

in order to form a prepolymer (P) containing at least one isocyanate function, preferably containing 2 isocyanate functions;

followed by a second step in which the prepolymer (P) obtained is reacted with a first alkoxysilane of formula (III) and a second alkoxysilane of formula (IV):

(R₁O)(R₂)(R₃)Si—CH₂—(NH-L₁)ₚ-X₁—H (III)

in which

p=0 or 1;

X₁ denotes —NRa—, S or O, Ra denoting H or a saturated or unsaturated C₁-C₈ (cyclo)alkyl radical, in particular methyl or cyclohexyl, or a C₆-C₁₀ aryl radical, in particular phenyl;

R₁ denotes a C₁-C₆ alkyl radical;

R₂ and R₃, which may be identical or different, preferably identical, are chosen from:
a C₁-C₆, in particular C₁-C₄, alkoxy radical;
a linear or branched C₁-C₆ alkyl radical;

L₁ denotes a linear or branched, saturated divalent hydrocarbon-based C₁-C₂₀ radical;

(R′₁O)(R′₂)(R′₃)Si—CH(R₄)—CH(R₅)-(L₂)_{q}-X₂—H (IV)

in which:

q=0 or 1;

X₂ denotes —NRb— or S or O or —NHCO—NRc-, Rb denoting H or a saturated or unsaturated C₁-C₈ (cyclo)alkyl radical, such as methyl, ethyl, butyl or cyclohexyl, or a C₆-C₁₀ aryl radical such as phenyl; Rc denoting a saturated C₁-C₄ alkyl radical, in particular methyl;

R′₁ denotes a C₁-C₆ alkyl radical;

R′₂ and R′₃, which may be identical or different, preferably identical are chosen from:
a C₁-C₆, in particular C₁-C₄, alkoxy radical;
a linear or branched C₁-C₆ alkyl radical;

R₅ denotes H or a C₁-C₄ alkyl radical optionally substituted with an —NH₂ group;

R₄ denotes H or a C₁-C₄ alkyl radical, in particular methyl;

L₂ denotes a linear or branched, saturated divalent hydrocarbon-based C₁-C₂₀ radical, optionally interrupted with an —NH— group, optionally substituted with an NH₂ group, it being possible for the mixture of the first and second alkoxysilanes (III) and (IV) to be added either simultaneously or sequentially by, for example, first introducing the first alkoxysiloxane (III) then the second alkoxysilane (IV), or else by first introducing the second alkoxysilane (IV) then the first alkoxysilane (III).

Advantageously, the reagents are used in the preparation process according to the following molar equivalents:

diisocyanate (I): 2 equivalents

difunctional compound (II): 1 equivalent

first alkoxysilane (III): u equivalent

second alkoxysilane (IV): v equivalent

with u+v=2 (u and v not being zero).

A subject of the invention is also the product (Pf) which can be obtained according to the preparation process described above.

For the diisocyanate (I) defined above, the Z radical is preferably chosen from the following radicals (1) to (6):

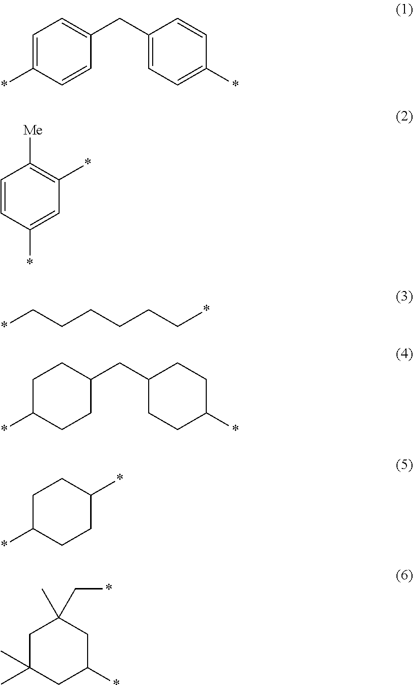

In the structures drawn, the * denotes the point of attachment with the isocyanate function. Preferentially, Z denotes the divalent radical isophorone (radical (6)).

For the difunctional compound of formula (II) defined above:

T preferably denotes O or NH;

A preferably denotes a linear or branched hydrocarbon-based C₂-C₅₀ radical optionally interrupted with one or more non-adjacent oxygen atoms.

In particular, A denotes a divalent radical chosen from:

(i) a divalent radical —[(CH₂)ₙ—O—]ₘ—(CH₂)_{n′}— with n and n′ independently denoting a number between 1 and 10 (limits included), preferably between 1 and 5, m denoting a number between 1 and 30, preferably between 1 and 20, more particularly between 1 and 10;

(ii) a C₂-C₅₀, preferably C₂₀-C₅₀, in particular C₃₂-C₄₀, alkylene radical, which is in particular non-linear, such as a C₃₆ radical;

(iii) a divalent radical —[CH(Me)-CH₂—O—]ₓ—(CH₂) CH(Me)- with x denoting a number between 2 and 70 (limits included), preferably between 2 and 7; in particular, x=2.5 or 6.1 or 33 or 68;

(iv) a divalent radical —CH(Me)-CH₂[O—CH₂—CH(Me)]_{x′}-[O—CH₂—CH₂]_{y′}[O—CH₂—CH(Me)-]_{z′} with y′ denoting a number between 2 and 50 (limits included), preferably between 2 and 40 and in particular y′=2 or 9 or 12.5 or 39, x′+z′ denoting a number between 1 and 10 (limits included), preferably between 1 and 7; in particular, x′+z′=1.2 or 3.6 or 6;

(v) a divalent radical —(CH₂)_{x″}—O—CH₂—CH₂—O—(CH₂)_{x″}— with x″ denoting a number between 1 and 10 (limits included), preferably between 1 and 5; in particular x″=2 or 3.

For the first alkoxysilane (III) defined above:

X₁ preferably denotes —NRa—, Ra being defined as above.

Preferentially, NRa denotes —NH— or —N-cyclohexyl.

R₁ preferably denotes a methyl or ethyl radical;

R₂ and R₃, which may be identical or different, preferably identical, are preferably chosen from:
methoxy or ethoxy radicals;
methyl or ethyl, preferably methyl, radicals.

Preferably, L₁ represents a linear or branched, saturated hydrocarbon-based C₁-C₁₀ radical, more particularly a saturated linear hydrocarbon-based C₁-C₁₀ radical.

According to one preferred embodiment, when p=1, L₁ represents a saturated divalent C₁-C₈ radical and in particular a divalent radical chosen from —CH₂—CH₂— and —(CH₂)₆—.

Advantageously, the first alkoxysilane (III) can be chosen from those of formula (IIIa) below:

(R₁O)(R₂)(R₃)Si—CH₂—(NH-L₁)ₚ-NRa—H (IIIa)

in which:

p=0 or 1;

R₁ denotes a methyl or ethyl radical; R₂ and R₃, which may be identical or different, denote a methoxy, ethoxy, methyl or ethyl radical;

when p=1, L₁ represents a saturated divalent hydrocarbon-based C₁-C₈ radical;

Ra denotes H or a saturated or unsaturated C₁-C₈ (cyclo)alkyl radical, or a phenyl radical;

preferably, Ra denotes H or a cyclohexyl radical.

As examples of a first alkoxysilane (III), mention may be made of:

| Chemical name | CAS No. | Chemical structure |
|---|---|---|
| 1-(dimethoxymethylsilyl)methanamine | (343926-26-1) | 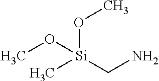 |
| 1-(diethoxymethylsilyl)methanamine | (18186-77-1) | 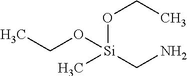 |
| 1-(triethoxysilyl)methanamine | (18306-83-7) | 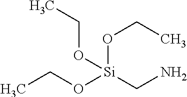 |
| 1-(trimethoxysilyl)methanamine | (71408-48-5) | 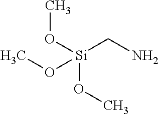 |
| 1-(trimethoxysilyl)methanethiol | (30817-94-8) | 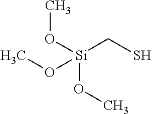 |
| 1-(diethoxymethylsilyl)methanethiol | (55161-63-2) | 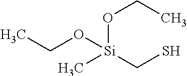 |
| 1-(triethoxysilyl)methanethiol | (60764-83-2) | 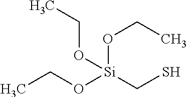 |
| 1-(triethoxysilyl)methanol | (162781-70-6) | 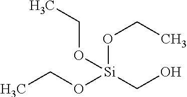 |
| N-[(triethoxysilyl)methyl]benzenamine | (3473-76-5) | 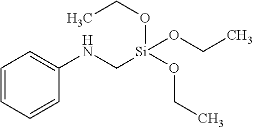 [SMILES: CCO[Si](CNc1ccccc1)(OCC)OCC] |
| N-[(trimethoxysilyl)methyl]benzenamine | (77855-73-3) | 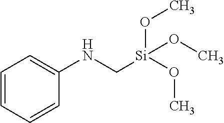 |
| N- [(diethoxymethylsilyl)methyl]cyclohexan- amine | 27445-54-1 | 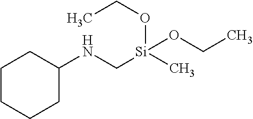 [SMILES: CCO[Si](C)(CNC1CCCCC1)OCC] |
| N- [(triethoxysilyl)methyl]cyclohexanamine | (26495-91-0) | 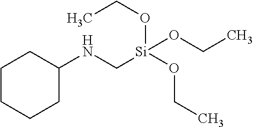 [SMILES: CCO[Si](CNC1CCCCC1)(OCC)OCC] |
| N-[(dimethoxymethylsilyl)methyl]- cyclohexanamine | (733051-93-9) | 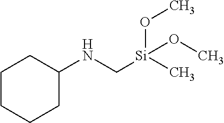 |
| N-(diethoxymethylsilyl)-N- methylmethanamine | (18306-82-6) | 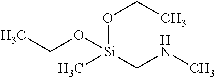 |
| N-methyl-1- (trimethoxysilyl)methanamine | (123271-16-9) | 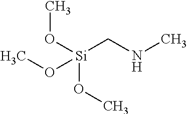 |
| N-methyl-1-(triethoxysilyl)methanamine | (151734-80-4) | 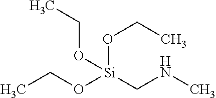 |
| N- [(dimethoxy(methyl)silyl)methyl]benzen- amine | (17890-10-7) | 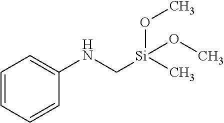 |
| N-[(triethoxysilyl)methyl]-1,6- hexanediamine | (15129-36-9) | 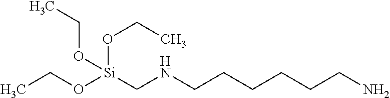 [SMILES: CCO[Si](CNCCCCCCN)(OCC)OCC] |
| N-[(trimethoxysilyl)methyl]-1,6- hexanediamine | (172684-43-4) | 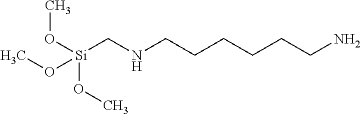 [SMILES: CO[Si](CNCCCCCCN)(OC)OC] |
| N-[(diethoxymethylsilyl)methyl]-1,6- hexanediamine | (15383-20-7) | 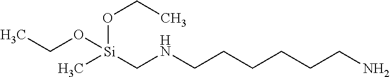 [SMILES: CCO[Si](C)(CNCCCCCCN)OCC] |
| N-[(trimethoxysilyl)methyl]-1,2- ethanediamine | (51980-40-6) | 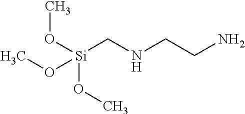 |

As examples of a preferred first alkoxysilane (III), mention may be made of:

1-(diethoxymethylsilyl)methanamine (18186-77-1)

1-(triethoxysilyl)methanamine (18306-83-7)

N-[(diethoxymethylsilyl)methyl]cyclohexanamine (27445-54-1)

N-[(triethoxysilyl)methyl]cyclohexanamine (26495-91-0)

N-[(triethoxysilyl)methyl]-1,6-hexanediamine (15129-36-9)

More preferentially, use is made, as first alkoxysilane (III), of

N-[(triethoxysilyl)methyl]-1,6-hexanediamine (15129-36-9)

N-[(triethoxysilyl)methyl]cyclohexanamine (26495-91-0).

For the second alkoxysilane (IV) defined above:

X₂ preferably denotes —NRb— or S, Rb denoting H or a saturated or unsaturated C₁-C₈ (cyclo)alkyl radical, such as methyl, ethyl, butyl or cyclohexyl, or a C₆-C₁₀ aryl radical such as phenyl. Preferentially, X₂ denotes —NH— or S.

Preferably, L₂ represents a linear or branched, saturated hydrocarbon-based C₁-C₁₂ radical, optionally interrupted with an —NH— group.

More preferentially, when q=1, L₂ represents a saturated divalent C₁-C₁₀ radical, or else a divalent radical —(CH₂)ₙ—NH—(CH₂)ₘ with n and m denoting integers such that 2≦n+m≦4,

and in particular a divalent radical chosen from —CH₂—, —CH₂—CH₂—, —(CH₃)(CH₃)C—CH₂—, —(CH₂)₉— and —CH₂—NH—O₂H₄—.

R′₁ preferably denotes a methyl or ethyl radical;

R′₂ and R′₃, which may be identical or different, preferably identical, are preferably chosen from:
methoxy or ethoxy radicals;
methyl or ethyl, preferably methyl, radicals.

R′₄ and R′₅ preferably denote H.

Advantageously, the second alkoxysilane (IV) can be chosen from those of formula (IVa) below:

(R′₁O)(R′₂)(R₃)Si—CH₂—CH₂-(L₂)_{q}-NRb—H (IVa)

in which:

q=0 or 1;

Rb denoting H or a saturated or unsaturated C₁-C₈ (cyclo)alkyl radical, such as methyl, ethyl, butyl or cyclohexyl, or a phenyl radical;

R′₁ denotes a methyl or ethyl radical; R′₂ and R′₃, which may be identical or different, denote a methoxy, ethoxy, methyl or ethyl radical;

L₂ denotes a linear or branched, saturated hydrocarbon-based C₁-C₁₂ radical, optionally interrupted with an —NH— group.

More preferentially, when q=1, L₂ represents a saturated divalent C₁-C₁₀ radical, or else a divalent radical —(CH₂)ₙ—NH—(CH₂)ₘ with n and m denoting integers such that 2≦n+m≦4, and in particular a divalent radical chosen from —CH₂—, —CH₂—CH₂—, —(CH₃)(CH₃)C—CH₂—, —(CH₂)₉— and —CH₂—NH—C₂H₄—; and more particularly a divalent radical chosen from —CH₂—, —CH₂—CH₂—, —(CH₃)(CH₃)C—CH₂— and —(CH₂)₉—.

As examples of a second alkoxysilane (IV), mention may be made of:

| Chemical name | CAS No. | Chemical structure |
|---|---|---|
| 3-(dimethoxymethylsilyl)-1- propanamine | (3663-44-3) | 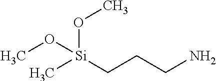 |
| 3-(trimethoxysilyl)-1-propanamine | (13822-56-5) | 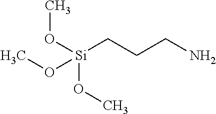 |
| 3-(triethoxysilyl)-1-propanamine | (919-30-2) | 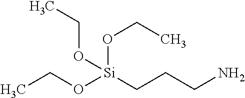 |
| 3-(diethoxymethoxysilyl)-1- propanamine | (61083-96-3) | 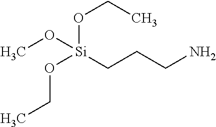 |
| 2-methyl-3-(trimethoxysilyl)-1- propanamine | (99503-87-4) | 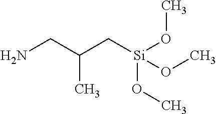 |
| 3-(triethoxysilyl)-1-propanamine | (29159-37-3) | 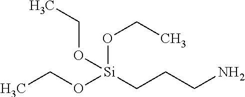 |
| 3-(diethoxymethylsilyl)-1- propanamine | (3179-76-8) | 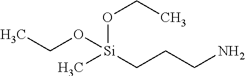 |
| 3-(methyldipropoxysilyl)-1- propanamine | (55081-09-9) | 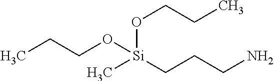 |
| 3-(diethoxyethylsilyl)-1- propanamine | (20723-29-9) | 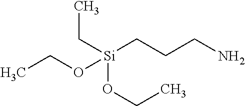 |
| 3-(ethyldimethoxysilyl)-1- propanamine | (639464-90-7) | 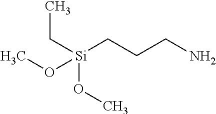 |
| 4-(triethoxysilyl)-1-butanamine | (3069-30-5) | 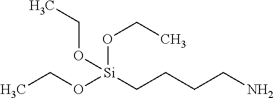 |
| 4-(dimethoxymethylsilyl)-1- butanamine | (18306-81-5) | 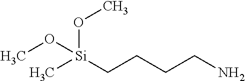 |
| 4-(trimethoxysilyl)-1-butanamine | (15005-59-1) | 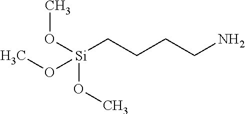 |
| 2,2-dimethyl-4-(trimethoxysilyl)-1- butanamine | (157923-74-5) | 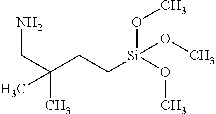 |
| 4-(diethoxymethylsilyl)-1- butanamine | (3037-72-7) | 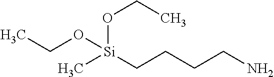 |
| 4-(dimethoxymethylsilyl)-2,2- dimethyl-1-butanamine | (156849-43-3) | 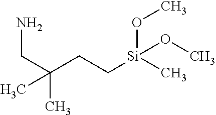 |
| 11-(triethoxysilyl)-1-undecamine | (116821-45-5) | 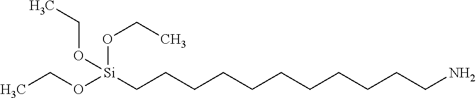 [SMILES: CCO[Si](CCCCCCCCCCCN)(OCC)OCC] |
| 11-(trimethoxysilyl)-1-undecamine | (40762-31-0) | 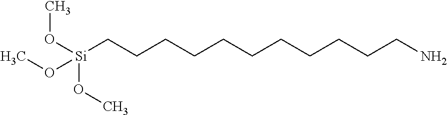 [SMILES: CO[Si](CCCCCCCCCCCN)(OC)OC] |
| 2-[(dimethoxymethylsilyl)methyl]- 1,4-butanediamine | (1019109-96-6) | 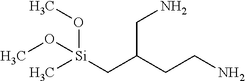 |
| 2-[(trimethoxysilyl)methyl]-1,4- butanediamine | (6037-49-6) | 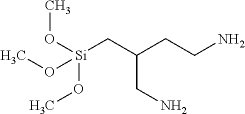 |
| N-(3- (trimethoxysilyl)propyl)butylamine | (31024-56-3) | 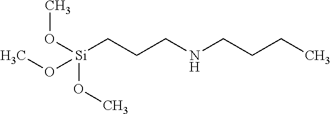 |
| N-ethyl-3-(trimethoxysilyl)-1- propanamine | (3451-81-8) | 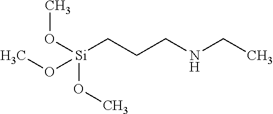 |
| N-methyl-3- (trimethoxysilyl)propylamine | (3069-25-8) | 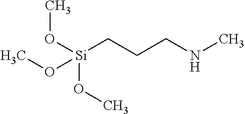 |
| N-[3- trimethoxysilyl]propyl]cyclohexyl- amine | (3068-78-8) | 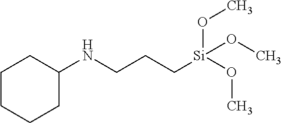 [SMILES: CO[Si](CCCNC1CCCCC1)(OC)OC] |
| N-[3-trimethoxysilyl]propyl]aniline | (3068-76-6) | 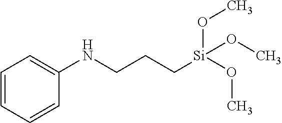 [SMILES: CO[Si](CCCNc1ccccc1)(OC)OC] |
| N-[3- trimethoxysilyl]propyl]ethylene- diamine | (1760-24-3) | 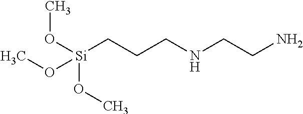 |
| N-[3-triethoxysilyl]propyl]ethylene- diamine | (5089-72-5) | 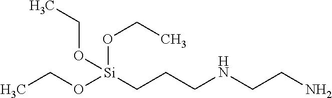 [SMILES: CCO[Si](CCCNCCN)(OCC)OCC] |
| 1-(trimethoxysilyl)-2-propanamine | (130530-83-5) | 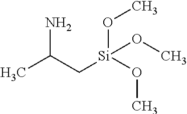 |
| 2-(trimethoxysilyl)ethanamine | (65644-31-7) | 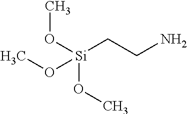 |
| 2-(triethoxysilyl)-1-propanamine | (36957-84-3) | 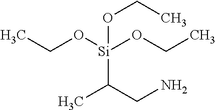 |
| 2- (dimethoxymethylsilyl)ethanamine | (115599-33-2) | 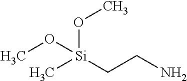 |
| 2-(diethoxymethylsilyl)-1- propanamine | (53813-15-3) | 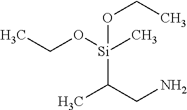 |
| 2-(diethoxymethylsilyl)ethanamine | (51250-43-2) | 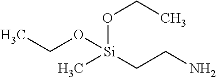 |
| 2-(triethoxysilyl)ethanamine | (45074-31-5) | 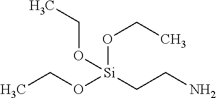 |
| 4-(trimethoxysilyl)-1-butanol | (177072-52-5) | 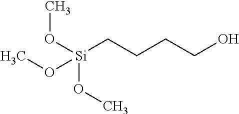 |
| 3-(trimethoxysilyl)-1-propanol | (53764-54-8) | 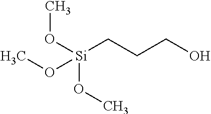 |
| 11-(trimethoxysilyl)-1- undecanethiol | (877593-17-4) | 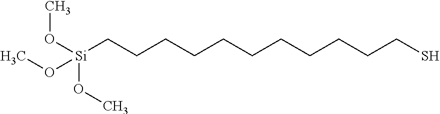 [SMILES: CO[Si](CCCCCCCCCCCS)(OC)OC] |
| 4-(trimethoxysilyl)-2-butanethiol | (57640-10-5) | 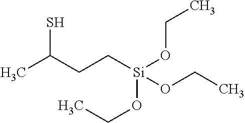 |
| 2-(triethoxysilyl)ethanethiol | (18236-15-2) | 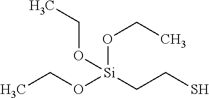 |
| 3-(triethoxysilyl)-1-propanethiol | (14814-09-6) | 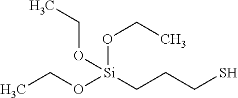 |
| 2-(trimethoxysilyl)ethanethiol | (7538-45-6) | 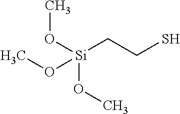 |
| 3-(trimethoxysilyl)-1-propanethiol | (4420-74-0) | 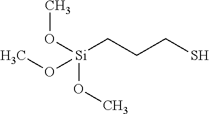 |
| 3-(dimethoxymethylsilyl)-1- propanethiol | (31001-77-1) | 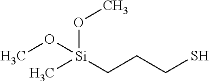 |
| N-[3- (trimethoxysilyl)propyl]acetamide | (57757-66-1) | 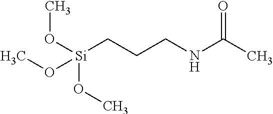 |

As examples of a preferred second alkoxysilane (IV), mention may be made of:

3-(triethoxysilyl)-1-propanamine (919-30-2)

3-(diethoxymethoxysilyl)-1-propanamine (61083-96-3)

3-(triethoxysilyl)-1-propanamine (29159-37-3)

3-(diethoxymethylsilyl)-1-propanamine (3179-76-8)

3-(diethoxyethylsilyl)-1-propanamine (20723-29-9)

4-(triethoxysilyl)-1-butanamine (3069-30-5)

4-(diethoxymethylsilyl)-1-butanamine (3037-72-7)

11-(triethoxysilyl)-1-undecamine (116821-45-5)

N-[3-triethoxysilyl]propyl]ethylenediamine (5089-72-5)

2-(triethoxysilyl)ethanethiol (18236-15-2)

3-(triethoxysilyl)-1-propanethiol (14814-09-6)

As particularly preferred alkoxysilane (IV), mention may be made of (3-aminopropyl)triethoxysilane and 3-(triethoxysilyl)-1-propanethiol.

In the process according to the invention, the first and second alkoxysilanes (III) and (IV) can be used in any relative proportions.

Preferably, the mixture of alkoxysilane (III) and (IV) used comprises from 5 to 95 mol % of alkoxysilane (III), relative to the total moles of alkoxysilanes (III) and (IV).

Preferentially, the mixture of alkoxysilane (III) and (IV) used comprises from 20 to 80 mol % of alkoxysilane (III), relative to the total moles of alkoxysilanes (III) and (IV).

In particular, the mixture of alkoxysilane (III) and (IV) used comprises from 30 to 70 mol % of alkoxysilane (III), relative to the total moles of alkoxysilanes (III) and (IV).

In particular, the mixture of alkoxysilane (III) and (IV) used comprises from 50 to 70 mol % of alkoxysilane (III), relative to the total moles of alkoxysilanes (III) and (IV).

It is understood that the amount of alkoxysilane (IV) in these mixtures is the remainder to 100 mol % adding to the molar amount of alkoxysilane (III) indicated.

As indicated above, in the second step of the process, the alkoxysilanes (III) and (IV) can be added simultaneously to the prepolymer (P) or else added sequentially by, for example, first adding the alkoxysilane (III) then the alkoxysilane (IV), or else first the alkoxysilane (IV) then the alkoxysilane (III).

In the preparation process described above, the first step can be carried out in the presence of a catalyst, in particular a tin-based organic catalyst, such as tin 2-ethylhexanoate, dibutyltin dilaurate, dioctyltin dilaurate, butyltin tris(2-ethylhexanoate), dibutyltin diacetate or dioctyltin diacetate, and preferably tin 2-ethylhexanoate.

Advantageously, the first step of the preparation process is carried out in an aprotic solvent, such as methyltetrahydrofuran, tetrahydrofuran or toluene, at a temperature of between 40° C. and 120° C., in particular between 50° C. and 70° C.

The first step may be carried out with a reaction time ranging from 3 to 7 hours.

The second step of the preparation process may be carried out at a temperature of between 20° C. and 60° C., in particular at ambient temperature (25° C.). This second step may be carried out with a reaction time ranging from 2 to 12 hours.

After the second step of the process, it is possible to carry out a solvent exchange, according to the techniques known to those skilled in the art, in particular by elimination of the aprotic solvent (gradual elimination by distillation) and addition of a carrier solvent of the obtained polymer comprising an alkoxysilane group (Pf). The carrier solvent may be an alcohol solvent, in particular a C₂-C₂₂ alcohol solvent, such as ethanol, isopropanol, propanol, t-butanol, sec-butanol or 2-octyldodecanol.

Advantageously, the obtained polymer comprising an alkoxysilane group (Pf) is carried in a carrier solvent, in particular an alcohol solvent as described above.

The preparation process described above can be represented schematically according to the following simplified reaction scheme.

First Step:

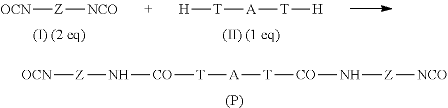

Second Step:

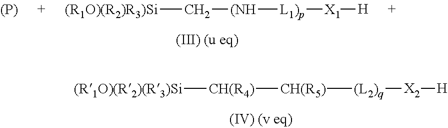

mixture containing the 3 compounds C1, C2, C3

(R₁O)(R₂)(R₃)Si—CH₂—(NH-L₁)ₚ-X₁—CO—NH—Z—NH—CO-T-A-T-CO—NH—Z—NH—CO—X₁-(L₁-NH—)ₚ-CH₂—Si (R₁O)(R₂)(R₃) (C1)

and

(R₁O)(R₂)(R₃)Si—CH₂—(NH-L₁)ₚ-X₁—CO—NH—Z—NH—CO-T-A-T-CO—NH—Z—NH—CO—X₂-(L₂)_{q}-CH(R₅)—CH(R₄)— . . . Si(R′₁O)(R′₂)(R′₃) (C2)

and

(R′₁O)(R′₂)(R′₃)Si—CH(R₄)—CH(R₅)-(L₂)_{q}-X₂—CO—NH—Z—NH—CO-T-A-T-CO—NH—Z—NH—CO—X₂-(L₂)-CH(R₅)—CH(R₄)— . . . —Si(R′₁O)(R′₂)(R′₃) (C3)

u denoting the number of molar equivalents of alkoxysilane (III) placed in reaction

v denoting the number of molar equivalents of alkoxysilane (IV) placed in reaction

with u+v=2, v other than 0.

Preferably, u is greater than 1.

According to one preferred embodiment of the preparation process, u is between 0.1 and 1.9 (limits included). Preferably, u is between 0.4 and 1.6. Preferentially, u is between 0.6 and 1.4. In particular, u is between 1 and 1.4.

The final product is obtained at the end of the reaction (total consumption of the isocyanate functions) in the form of a solution in a solvent which may be the reaction solvent or a carrier solvent, such as an alcohol solvent, in particular by solvent exchange, as described above.

The simplified reaction scheme described above is an illustration of the case corresponding to the formation of the pure prepolymer (P). Nevertheless, the prepolymer (P) can be obtained as a mixture with other compounds resulting from the condensation of (I) with (II) and/or (II) with (P); thus, it is possible to obtain, in the final product (Pf), other compounds which are additional to the compounds C1, C2 and C3, resulting in particular from the polycondensation of the compound (P) with the compound (II), then of these products with the compounds (III) and/or (IV).

A subject of the invention is the product which is a polymer comprising an alkoxysilane group (Pf), which can be obtained with the preparation process described above.

As indicated above, the preparation process makes it possible to obtain a mixture comprising the compounds C1, C2 and C3 described above.

Thus, a subject of the invention is the mixture of the compounds C1, C2 and C3.

A further subject of the invention is the compound C2 as novel compound and for which T denotes O or NH; A denotes a linear or branched hydrocarbon-based C₂-C₅₀ radical optionally interrupted with one or more non-adjacent oxygen atoms.

A further subject of the invention is the compound C3 as novel compound.

A further subject of the invention is an anhydrous composition comprising, in a physiologically acceptable medium, a product or compound or mixture of compounds as defined above. The term “physiologically acceptable medium” is intended to mean a medium that is compatible with keratin materials such as the skin, the hair or the nails, as a cosmetic medium.

In particular, the composition comprises the product (Pf) obtained according to the preparation process described above.

In particular, the composition comprises a mixture of the compounds C1, C2 and C3 as described above.

The product (Pf) or the mixture of compounds comprising C1, C2, C3 may be present in the composition according to the invention in a content ranging from 0.1% to 60% by weight, relative to the total weight of the composition, preferably ranging from 0.1% to 50% by weight, preferentially ranging from 0.5% to 45% by weight.

A further subject of the invention is a process, in particular a cosmetic process, for caring for or making up keratin materials, in particular the nails or the hair or the skin, comprising the application to the keratin materials, in particular to the nails or the hair or the skin, of a composition as described above.

According to one particular embodiment, the composition according to the present invention may also comprise at least one volatile organic solvent.

The term “volatile organic solvent” denotes, in the present invention, an organic compound which is liquid at ambient temperature (25° C.), which comprises at least one group chosen from hydroxyl, ester, ketone, ether or aldehyde groups, and which has a vapour pressure greater than 1 mbar (100 Pa) at 20° C.

Among the volatile organic solvents that may be used in the composition in accordance with the invention, mention may be made of lower monoalcohols containing from 1 to 5 carbon atoms, such as ethanol and isopropanol, C₃-C₄ ketones, C₂-C₄ aldehydes and C₂-C₄ short-chain esters.

The composition according to the invention is anhydrous. The term “anhydrous” is intended to mean a composition comprising a content of less than or equal to 2% and in particular 1% by weight of water, relative to the total weight of the composition, or is even free of water. It is in particular intended to mean that water is preferably not deliberately added to the composition, but may be present in trace amounts in the various compounds used in the composition.

The composition according to the invention may also comprise a cosmetic adjuvant chosen from film-forming polymers, plasticizers, colorants, preservatives, fragrances, fillers, oils, waxes, thickeners, antioxidants, surfactants and skin care active agents.

The invention will now be described with reference to the examples that follow, which are given as non-limiting illustrations.

### EXAMPLE 1

22.2 g (0.10 mol) of isophorone diisocyanate, 100 μl of tin 2-ethylhexanoate catalyst and 270 g of methyltetrahydrofuran (MeTHF) (dried over sieve) are introduced into a 500 ml reactor equipped with a dropping funnel, under an argon atmosphere. The solution was heated to 55° C. 26.7 g (0.05 mol) of C₃₆ non-linear diol dimer (Pripol® 2033 from Croda) diluted in 20 g of MeTHF were then added over the course of 40 minutes. At the end of the addition, heating was carried out at 65° C. until half the isocyanate functions had been consumed.

20.5 g (0.07 mol) of N-(6-aminohexyl)aminomethyltriethoxysilane in 30 g of MeTHF were introduced into the dropping funnel and then this mixture was added to the reactor at ambient temperature, over the course of 30 min. 6.6 g (0.03 mol) of (3-aminopropyl) triethoxysilane diluted in 10 g of MeTHF were then added dropwise. Reaction was allowed to take place for 4 hours and then 200 g of isopropanol were added and the resulting mixture was heated for 1 hour at 60° C.; the temperature was then increased in order to distill off the MeTHF. When half the amount of MeTHF had been distilled off, 100 g of isopropanol were added and the distillation was continued. The operation was repeated until the MeTHF had been totally removed. In the end, a solution containing a mixture of compounds (Pf) with a solids content of 60% by weight in isopropanol was obtained.

The solution obtained contains the following compounds:

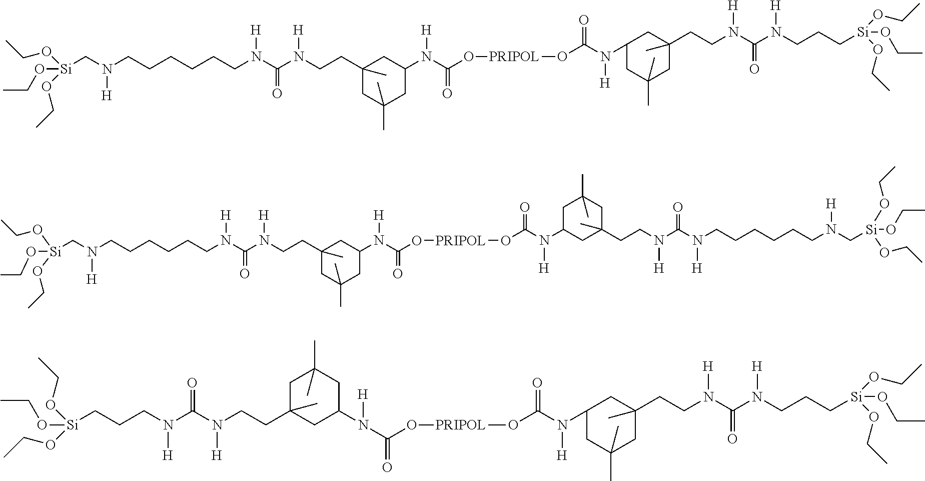

with Pripol denoting the non-linear divalent C₃₆ radical of the dimer diol Pripol 2033.

The solution obtained, comprising the mixture of these compounds, applied to a Teflon® plate, rapidly forms a film. The film obtained is uniform, transparent, shiny and non-tacky. The film obtained was subsequently detached from the plate and then placed in a crystallizer filled with water and with stirring for 24 hours at 25° C.: after this time, it was noted that the film remains in a state that is still transparent and shiny and therefore has good water resistance.

### EXAMPLE 2

A nail varnish having the following composition (as weight percentage) is prepared:

| | |
|---|---|
| solution obtained according to Example 1 | 99% |
| Red 27 pigment | 1% |

The varnish composition, after application to false nails, forms, on contact with the air, a uniform, glossy and water- and scratch-resistant film.

### EXAMPLE 3

A hair composition as follows, packaged in a pump dispenser bottle, is prepared:

| | |
|---|---|
| solution obtained according to Example 1 | 1% AM |
| ethanol | qs 100% |

After the application of the composition to the hair, the latter is shiny and also has more body (it is not lank). It is easier to style.

### EXAMPLE 4

A skincare composition as follows, packaged in a pump dispenser bottle, is prepared:

| | |
|---|---|
| solution obtained according to Example 1 | 3% AM |
| 2-octyldodecanol | qs 100% |

A few drops of the composition are deposited on the finger and the product is then applied to the wrinkled area of the face. After application, the deposit formed fills the relief of the treated skin, and the area treated appears smoother.

1. A process for preparing a polymer comprising alkoxysilane groups which can be obtained by polycondensation, comprising, in a first step, the reaction between:
(i) a diisocyanate of formula (I): OCN—Z—NCO (I)
in which Z denotes a divalent hydrocarbon-based radical containing from 4 to 20 carbon atoms; and
(ii) a difunctional compound of formula (II): H-T-A-T-H (II)
in which:
T denotes a heteroatom chosen from O and S or an —N(R)— radical, R being H or a C₁-C₄ alkyl radical,
A denotes a linear or branched, divalent hydrocarbon-based C₂-C₁₀₀ radical, optionally interrupted with one or more non-adjacent heteroatoms chosen from O and S, or an —N(R′)— group in which R′ denotes a hydrogen atom or a C₁-C₄ alkyl radical; in order to form a prepolymer (P) containing at least one isocyanate function;
followed by a second step in which the prepolymer (P) obtained is reacted with a first alkoxysilane of formula (III) and a second alkoxysilane of formula (IV):
(R₁O)(R₂)(R₃)Si—CH₂—(NH-L₁)ₚ-X₁—H (III)
in which
p=0 or 1;
X₁ denotes —NRa—, S or O, Ra denoting H or a saturated or unsaturated C₁-C₈ (cyclo)alkyl radical, in particular methyl or cyclohexyl, or a C₆-C₁₀ aryl radical;
R₁ denotes a C₁-C₆ alkyl radical;
R₂ and R₃, which may be identical or different, preferably identical are chosen from:
a C₁-C₆, in particular C₁-C₄, alkoxy radical;
a linear or branched C₁-C₆ alkyl radical;
L₁ denotes a linear or branched, saturated divalent hydrocarbon-based C₁-C₂₀ radical;
(R′₁O)(R′₂)(R′₃)Si—CH(R₄)—CH(R₅)-(L₂)_{q}-X₂—H (IV)
in which:
q=0 or 1;
X₂ denotes —NRb— or S or O or —NHCO—NRc-, Rb denoting H or a saturated or unsaturated C₁-C₈ (cyclo)alkyl radical, or a C₆-C₁₀ aryl radical; Rc denoting a saturated C₁-C₄ alkyl radical;
R′₁ denotes a C₁-C₆ alkyl radical;
R′₂ and R′₃, which may be identical or different, are chosen from:
a C₁-C₆;
a linear or branched C₁-C₆ alkyl radical;
R₄ denotes H or a C₁-C₄ alkyl radical;
R₅ denotes H or a C₁-C₄ alkyl radical optionally substituted with an —NH₂ group;
L₂ denotes a linear or branched, saturated divalent hydrocarbon-based C₁-C₂₀ radical, optionally interrupted with an —NH— group, optionally substituted with an NH₂ group;
it being possible for the first and second alkoxysilanes (III) and (IV) to be added either simultaneously or sequentially by first introducing the first alkoxysiloxane (III) then the second alkoxysilane (IV), or by first introducing the second alkoxysilane (IV) then the first alkoxysilane (III).

2. The process according to claim 1, wherein, for the diisocyanate (I), the Z radical is chosen from the following radicals (1) to (6):

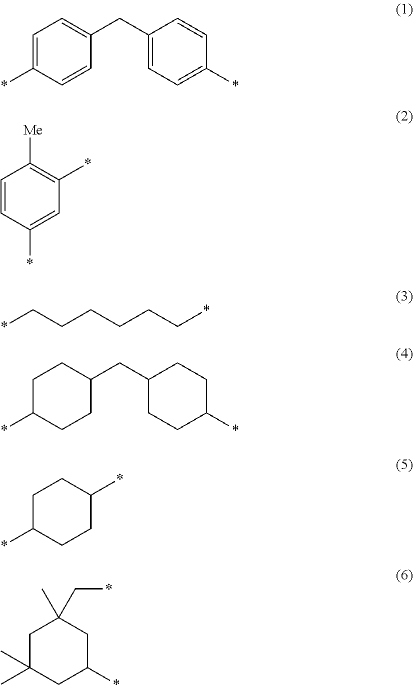

3. The process according to claim 1, wherein, for the difunctional compound of formula (II):
T denotes O or NH;
A denotes a linear or branched hydrocarbon-based C₂-C₅₀ radical optionally interrupted with one or more non-adjacent oxygen atoms.

4. The process according to claim 1, wherein, for the first alkoxysilane (III):
X₁ denotes —NRa;
R₁ denotes a methyl or ethyl radical;
R₂ and R₃, which may be identical or different, are chosen from:
methoxy or ethoxy radicals;
methyl or ethyl;
L₁ represents a linear or branched, saturated hydrocarbon-based C₁-C₁₀ radical.

5. The process according to claim 1, wherein the first alkoxysilane (III) is chosen from those of formula (IIIa) below:
(R₁O)(R₂)(R₃)Si—CH₂—(NH-L₁)ₚ-NRa—H (IIIa)
in which:
p=0 or 1;
R₁ denotes a methyl or ethyl radical; R₂ and R₃, which may be identical or different, denote a methoxy, ethoxy, methyl or ethyl radical;
when p=1, L₁ represents a saturated divalent hydrocarbon-based C₁-C₈ radical;
Ra denotes H or a saturated or unsaturated C₁-C₈ (cyclo)alkyl radical, or a phenyl radical;
preferably, Ra denotes H or a cyclohexyl radical.

6. The process according to claim 1, wherein the first alkoxysilane (III) is chosen from:
1-(dimethoxymethylsilyl)methanamine
1-(diethoxymethylsilyl)methanamine
1-(triethoxysilyl)methanamine
1-(trimethoxysilyl)methanamine
1-(trimethoxysilyl)methanethiol
1-(diethoxymethylsilyl)methanethiol
1-(triethoxysilyl)methanethiol
1-(triethoxysilyl)methanol
N-[(triethoxysilyl)methyl]benzenamine
N-[(trimethoxysilyl)methyl]benzenamine
N-[(diethoxymethylsilyl)methyl]cyclohexanamine
N-[(triethoxysilyl)methyl]cyclohexanamine
N-[(dimethoxymethylsilyl)methyl]-cyclohexanamine
N-(diethoxymethylsilyl)-N-methylmethanamine
N-methyl-1-(trimethoxysilyl)methanamine
N-methyl-1-(triethoxysilyl)methanamine
N-[(dimethoxy(methyl)silyl)methyl]benzenamine
N-[(triethoxysilyl)methyl]-1,6-hexanediamine
N-[(trimethoxysilyl)methyl]-1,6-hexanediamine
N-[(diethoxymethylsilyl)methyl]-1,6-hexanediamine
N-[(trimethoxysilyl)methyl]-1,2-ethanediamine.

7. The process according to claim 1, wherein, for the second alkoxysilane (IV):
X₂ denotes —NRb— or S, Rb denoting H or a saturated or unsaturated C₁-C₈ (cyclo)alkyl radical, or a C₆-C₁₀ aryl radical;
L₂ represents a linear or branched, saturated hydrocarbon-based C₁-C₁₂ radical, optionally interrupted with an —NH— group; preferably, when q=1, L₂ represents a saturated divalent C₁-C₁₀ radical, or else a divalent radical —(CH₂)ₙ—NH—(CH₂)ₘ with n and m denoting integers such that 2≦n+m≦4;
R′₁ denotes a methyl or ethyl radical,
R′₂ and R′₃, which may be identical or different, are chosen from:
methoxy or ethoxy radicals;
methyl or ethyl;
R′₄ and R′₅ denote H.

8. The process according to claim 1, wherein
the second alkoxysilane (IV) is chosen from those of formula (IVa) below:
(R′₁O)(R′₂)(R₃)Si—CH₂—CH₂-(L₂)_{q}-NRb—H (IVa)
in which:
q=0 or 1;
Rb denoting H or a saturated or unsaturated C₁-C₈ (cyclo)alkyl radical;
R′₁ denotes a methyl or ethyl radical; R′₂ and R′₃, which may be identical or different, denote a methoxy, ethoxy, methyl or ethyl radical;
L₂ denotes a linear or branched, saturated hydrocarbon-based C₁-C₁₂ radical, optionally interrupted with an —NH— group; when q=1, L₂ represents a saturated divalent C₁-C₁₀ radical, or else a divalent radical —(CH₂)—NH—(CH₂)ₘ with n and m denoting integers such that 2≦n+m≦4.

9. The process according to claim 1, wherein the second alkoxysilane (IV) is chosen from:
3-(dimethoxymethylsilyl)-1-propanamine
3-(trimethoxysilyl)-1-propanamine
3-(triethoxysilyl)-1-propanamine
3-(diethoxymethoxysilyl)-1-propanamine
2-methyl-3-(trimethoxysilyl)-1-propanamine
3-(triethoxysilyl)-1-propanamine
3-(diethoxymethylsilyl)-1-propanamine
3-(methyldipropoxysilyl)-1-propanamine
3-(diethoxyethylsilyl)-1-propanamine
3-(ethyldimethoxysilyl)-1-propanamine
4-(triethoxysilyl)-1-butanamine
4-(dimethoxymethylsilyl)-1-butanamine
4-(trimethoxysilyl)-1-butanamine
2,2-dimethyl-4-(trimethoxysilyl)-1-butanamine
4-(diethoxymethylsilyl)-1-butanamine
4-(dimethoxymethylsilyl)-2,2-dimethyl-1-butanamine
11-(triethoxysilyl)-1-undecamine
11-(trimethoxysilyl)-1-undecamine
2-[(dimethoxymethylsilyl)methyl]-1,4-butanediamine
2-[(trimethoxysilyl)methyl]-1,4-butanediamine
N-(3-(trimethoxysilyl)propyl)butylamine
N-ethyl-3-(trimethoxysilyl)-1-propanamine
N-methyl-3-(trimethoxysilyl)propylamine
N-[3-trimethoxysilyl]propyl]cyclohexylamine
N-[3-trimethoxysilyl]propyl]aniline
N-[3-trimethoxysilyl]propyl]ethylenediamine
N-[3-triethoxysilyl]propyl]ethylenediamine
1-(trimethoxysilyl)-2-propanamine
2-(trimethoxysilyl)ethanamine
2-(triethoxysilyl)-1-propanamine
2-(dimethoxymethylsilyl)ethanamine
2-(diethoxymethylsilyl)-1-propanamine
2-(diethoxymethylsilyl)ethanamine
2-(triethoxysilyl)ethanamine
4-(trimethoxysilyl)-1-butanol
3-(trimethoxysilyl)-1-propanol
11-(trimethoxysilyl)-1-undecanethiol
4-(trimethoxysilyl)-2-butanethiol
2-(triethoxysilyl)ethanethiol
3-(triethoxysilyl)-1-propanethiol
2-(trimethoxysilyl)ethanethiol
3-(trimethoxysilyl)-1-propanethiol
3-(dimethoxymethylsilyl)-1-propanethiol
N-[3-(trimethoxysilyl)propyl]acetamide.

10. The process according to claim 1, wherein the mixture of first and second alkoxysilanes (III) and (IV) used comprises from 5 to 95 mol % of alkoxysilane (III), relative to the total moles of alkoxysilanes (III) and (IV).

11. The process according to claim 1, wherein the reagents are used according to the following molar equivalents:
diisocyanate (I): 2 equivalents
difunctional compound (II): 1 equivalent
first alkoxysilane (III): u equivalent
second alkoxysilane (IV): v equivalent
with u+v=2, u and v not being zero.

12. The process according to claim 1, wherein the first step is carried out in the presence of a catalyst, in particular a tin-based organic catalyst.

13. The process according to claim 1, wherein the first step is carried out in an aprotic solvent at a temperature of between 40° C. and 120° C.

14. The process according to claim 1, wherein the second step is carried out at a temperature of between 20° C. and 60° C.

15. The process according to claim 1, wherein the second step is followed by a step of solvent exchange by elimination of the aprotic solvent and addition of a carrier solvent.

16. The process according to claim 1, wherein the obtained polymer comprising an alkoxysilane group is carried in a carrier solvent.

17. A product which is a polymer comprising an alkoxysilane group (Pf), which can be obtained with the preparation process according to claim 1.

18. A mixture of compounds C1, C2 and C3:
(R₁O)(R₂)(R₃)Si—CH₂—(NH-L₁)ₚ-X₁—CO—NH—Z—NH—CO-T-A-T-CO—NH—Z—NH—CO—X₁-(L₁-NH—)ₚ—CH₂—Si(R₁O)(R₂)(R₃) (C1)
(R₁O)(R₂)(R₃)Si—CH₂—(NH-L₁)ₚ-X₁—CO—NH—Z—NH—CO-T-A-T-CO—NH—Z—NH—CO—X₂-(L₂)_{q}-CH(R₅)—CH(R₄)— . . . Si(R′₁O)(R′₂)(R′₃) (C2)
(R′₁O)(R′₂)(R′₃)Si—CH(R₄)—CH(R₅)-(L₂)_{q}-X₂—CO—NH—Z—NH—CO-T-A-T-CO—NH—Z—NH—CO—X₂-(L₂)_{q}-CH(R₅)—CH(R₄)— . . . —Si(R′₁O)(R′₂)(R′₃) (C3)

19. A compound of formula C2:
(R₁O)(R₂)(R₃)Si—CH₂—(NH-L₁)ₚ-X₁—CO—NH—Z—NH—CO-T-A-T-CO—NH—Z—NH—CO—X₂-(L₂)_{q}-CH(R₅)—CH(R₄)— . . . Si(R′₁O)(R′₂)(R′₃) (C2)
in which Z, R₁, R₂, R₃, R′₁, R′₂, R′₃, R₄, R₅, L₁, L₂, X₁, X₂, p and q have the meanings defined in claim 1, and
T denotes O or NH;
A denotes a linear or branched hydrocarbon-based C₂-C₅₀ radical optionally interrupted with one or more non-adjacent oxygen atoms.

20. An anhydrous composition comprising, in a physiologically acceptable medium, a product or compound(s) as defined according to claim 17.

21. A composition according to claim 20, wherein the product or the compounds are present in a content ranging from 0.1% to 60% by weight, relative to the total weight of the composition.

22. The composition according to claim 20, wherein it comprises at least one volatile organic solvent.

23. A cosmetic process for caring for or making up keratin materials, in particular the nails or the hair or the skin, comprising the application to the keratin materials, in particular to the nails or the hair or the skin, of a composition according to claim 21.
